(19) United States
(12) Patent Application Publication
(10) Pub. No.: US 2019/0142733 A1
(43) Pub. Date: May 16, 2019

(54) Activated Charcoal Formulation

(71) Applicant: Justin Arman, New Braunfels, TX (US)

(72) Inventor: Justin Arman, New Braunfels, TX (US)

(21) Appl. No.: 16/185,683

(22) Filed: Nov. 9, 2018

Related U.S. Application Data
(60) Provisional application No. 62/583,826, filed on Nov. 9, 2017.

(51) Int. Cl. A61K 8/97; A61Q 11/00; A61K 35/10

(52) U.S. Cl. CPC A61K 8/97; A61Q 11/00; A61K 2800/92; A61K 2800/42; A61K 35/10

(57) ABSTRACT

An aqueous based formulation for a personal care product, that relies upon the use of fulvic-humic acid and/or humic acid, which acid(s) are present at a level of 0.01 wt. % to 10.0 wt. % and activated carbon black at a level of ≤5.0 wt. %.

### CROSS REFERENCE TO RELATED APPLICATION

This application claims the benefit of U.S. Provisional Application Ser. No. 62/583,826 filed Nov. 9, 2017, the teachings of which are incorporated herein by reference.

### FIELD

The present invention relates to an aqueous based activated charcoal formulation. The aqueous based activated charcoal formulation is one that allows for relatively lower levels of charcoal to be employed that reduces the tendency of the charcoal to coagulate, clump and settle, while still providing the therapeutic effects of the charcoal ingredient.

### BACKGROUND

There are presently on the market a variety of personal care products that utilize activated charcoal in one form or another. For example, in WO2017/068546 entitled Composition For Oral Hygiene there is disclosed a composition for oral hygiene, in particular a toothpaste, that includes the use of activated charcoal. In addition, reference is made to the use of ultra-ventilated green clay and white clay. The activated charcoal is described as being present at a level of 1% to 30% by weight.

Reference is also made to U.S. Publication No. 2017/0020802 entitled Natural Composition. The composition disclosed is described to contain a plurality of ingredients that are mixed in predetermined percentages to formulate what is termed as a natural, healthful, and flavorful composition that integrates into at least one consumable item to produce a flavorful, healthful enhanced consumable item. The ingredients of the composition include a Frankincense oil, a gum, and a fruit aroma compound. The ingredients are mixed together in varying weight percentages for a desired outcome when integrated with the consumable item. The combination of ingredients is said to create a synergy that enhances the consumable item. The composition is mixed, dissolved, distilled, or ground with the consumable item. The consumable item may include a tobacco, a charcoal briquette, an energy drink, mineral water, butter, a mouthwash, and toothpaste.

### DETAILED DESCRIPTION

The present invention is directed at an aqueous based formulation that provides for the use of relatively lower levels of activated charcoal (carbon black) in a given personal care item, which reduces or avoids the tendency of the charcoal to coagulate, clump and/or settle in the formulation. The aqueous based formulation is preferably employed in the form of an oral rinse but it can be appreciated that other personal care products may be similarly prepared wherein relatively lower levels of charcoal may be utilized, without compromising the effectiveness of the charcoal additive.

By way of example, an oral rinse product has been prepared which is an aqueous base formulation that includes activated charcoal (carbon black) and the use of fulvic-humic acid powder and/or humic acid which acids appear to, among other things, provide a coloring influence and lead to a dark or black colored solution. Fulvic acids are understood herein as a family of organic acids that occur at relatively low levels in solid and water in nature. Humic substances are present in nature and arise from the decay of plant and animal residues in the environment. These substances can be divided into humic acid and fulvic acid on the basis of their solubility in water as a function of pH. Fulvic acid is the fraction of humic substances that is soluble in water under all pH conditions and is in general lower in molecular size and weight and lower in colour intensity than humic acid. The fulvic-humic acids that are employed herein are preferably sourced from natural organic compounds.

The level of fulvic-humic acid and/or the level of humic acid in the aqueous based formulation preferably falls in the range of 0.01 wt. % to 10.0 wt. %, more preferably 0.01 wt. % to 5.0 wt. %, and even more preferably, 0.01 wt. % to 3.0 wt. %. In a most preferred embodiment the level of fulvic-humic acid and/or the level of humic acid is 0.01 to 0.10 wt %, or in the range of 0.03 wt % to 0.09 wt. %.

Accordingly, in a first preferred embodiment a water soluble fulvic-humic acid powder is combined with activated charcoal (carbon) powder at an acid concentration level of 10.0 wt. % or below, or in the range of 0.01 wt. % to 10.0 wt. %. The humic acid is also preferably 51.0% of the water soluble fulvic-humic acid blend powder with other preferred proportions at (3:2, 4:1, 5:1) respectively. As alluded to above, it has been observed that the presence of such fulvic-humic acid turns the water base of the mouthwash dark brown to black in color while allowing for use of relatively low concentrations of insoluble activated charcoal (carbon) to provide the same therapeutic effect. This is contemplated to include anti-inflammatory benefits and reduction in gum disease, bone loss and decay. In addition, the formulation herein is one that can avoid the use of alcohol and fluoride while still provide what is contemplated to be an effective oral rinse protocol.

Preferably, the relatively low level of activated carbon black is such that it is present at levels of ≤5.0 wt. % and in the range of 1.0×10⁻⁵ wt. % to 5.0 wt. %. More preferably, the relatively low level of activated carbon black is such that it is in the range of 1.0×10⁻⁵ wt. % to 4.0 wt. %, or 1.0×10⁻⁵ wt. % to 3.0 wt. %, or 1.0×10⁻⁵ wt. % to 2.0 wt. %, or 1.0×10⁻⁵ wt. % to 1.0 wt. %. The level of activated carbon black may also fall in the range of 1.0×10⁻⁵ wt. % to 1.0×10⁻⁴ wt. % (i.e. 0.000010 wt. % to 0.00010 wt. %). One particularly preferred range is 2.0×10⁻⁵ wt. % (i.e. 0.000021 wt. %) to 5.0×10⁻⁵ wt. % (i.e. 0.000050 wt. %).

In the context of the present invention, it should be noted that 100% soluble humic acid powder may also be used instead of the humic-fulvic powder combination noted above. In either case, i.e. the use of the fulvic-humic acid blend and/or the use of the humic acid, such has been shown to provide a natural black dye to the aqueous formulation, along with the therapeutic effects of the acids, such that at noted, relatively lower amounts of carbon black can be utilized which then avoids coagulation of the carbon black and lumps of charcoal, and settling of the charcoal, that is undesirable.

Preferably, the charcoal or carbon black here is activated carbon black or activated charcoal, which is reference to a form of carbon that is processed to increase the number of pores that result in an increase in surface area. For example, one gram of activated carbon black herein can have a surface area of equal to or greater than 3000 m² as determined by gas absorption.

One exemplary preferred mouthwash formulation is provided below in Table 1 which is for a peppermint flavored oral rinse formulation:

TABLE 1

| Materials | Wt/Vol | | Target % | |
|---|---|---|---|---|
| RO Water | 346 | ml | 82.46 | Base |
| Xylitol | 50.15 | g | 10.6 | Sweetner |
| Aloe Vera Juice (Aloe | 1 | oz | 6.25 | Flavor |
| Barbadensis) | | | | |
| Sodium Bicarbonate | 1 | g | 0.21 | pH Stabilizer |
| Peppermint Flavor Powder | 0.6 | g | 0.126 | Flavor |
| Peppermint Flavor Powder | 0.6 | g | 0.126 | Flavor |
| Fulvic/Humic Acid | 0.3 | g | 0.063 | Color Agent |
| Turmeric (Corcuma | 0.256 | g | 0.054 | Extract |
| Longa) Extract | | | | |
| Menthol Flavor | 0.1 | g | 0.021 | Flavour |
| Vitamin C | 0.1 | g | 0.021 | Antioxidant |
| Nisin | 0.1 | g | 0.021 | Antimicrobial |
| Natamycin | 0.06 | g | 0.012 | Antifungal |
| Activated Coconut | 0.0001 | g | 0.000021 | Carbon black |
| Shell Charcoal | | | | |

In yet another exemplary embodiment shown in Table 2, a cinnamon rinse was prepared according to the following:

TABLE 2

| Materials | Wt/Vol | | Target % | |
|---|---|---|---|---|
| RO Water | 346 | ml | 82.46 | Base |
| Xylitol | 50.15 | g | 10.6 | Sweetner |
| Aloe Vera Juice (Aloe | 1 | oz | 6.25 | Flavor |
| Barbadensis) | | | | |
| Cinnamon Clove Flavour | 3.25 | g | 0.687 | Flavor |
| Sodium Bicarbonate | 1 | g | 0.21 | pH Stabilizer |
| Fulvic/Humic Acid | 0.3 | g | 0.063 | Color Agent |
| Vitamin C | 0.1 | g | 0.021 | Extract |
| Nisin | 0.1 | g | 0.021 | Antimicrobial |
| Natamycin | 0.06 | g | 0.012 | Antifungal |
| Menthol Flavor | 0.02 | g | 0.0042 | Flavor |
| Turmeric (Corcuma | 0.0125 | g | 0.0026 | Extract |
| Longa) Extract | | | | |
| Activated Coconut | 0.0001 | g | 0.000021 | Ingredient |
| Shell Charcoal | | | | |

As noted, the aqueous based formulations herein containing the presence of fulvic-humic acids and/or humic acids may preferably be utilized for a mouthwash formulation. However, it is contemplated herein that such aqueous based formulation can be used in a variety of other aqueous based personal care products. These may include, but not be limited to, skin creams, body wash, liquid soap, shampoos and conditioners, cleaner, face wash and shave gel.

As can now be seen from the above, the present invention is directed at an aqueous based formulation for a personal care product, that relies upon the use of fulvic-humic acid and/or humic acid, which acid(s) are present at a level of 0.01 wt. % to 10.0 wt. % and activated carbon black at a level of ≤5.0 wt. %.

1. An aqueous based formulation comprising:
0.01 wt. % to 10.0 wt. % fluvic-humic acid; and
≤5.0 wt. % of activated carbon black.

2. The aqueous based formulation of claim 1 wherein said fluvic-humic acid is present at a level of 0.01 wt. % to 5.0 wt. %.

3. The aqueous based formulation of claim 1 wherein said fluvic-humic acid is present at a level of 0.01 wt. % to 3.0 wt. %.

4. The aqueous based formulation of claim 1 wherein said fluvic-humic acid is present at a level of 0.01 wt. % to 0.10 wt. %.

5. The aqueous based formulation of claim 1 wherein said fluvic-humic acid is present at a level of 0.03 wt. % to 0.09 wt. %.

6. The aqueous based formulation of claim 1 wherein said carbon black is present at a level of 1.0×10⁻⁵ wt. % to 5.0 wt. %.

7. The aqueous based formulation of claim 1 wherein said carbon black is present at a level of 1.0×10⁻⁵ wt. % to 4.0 wt. %.

8. The aqueous based formulation of claim 1 wherein said carbon black is present at a level of 1.0×10⁻⁵ wt. % to 3.0 wt. %.

9. The aqueous based formulation of claim 1 wherein said carbon black is present at a level of 1.0×10⁻⁵ wt. % to 2.0 wt. %.

10. The aqueous based formulation of claim 1 wherein said carbon black is present at a level of 1.0×10⁻⁵ wt. % to 1.0 wt. %.

11. The aqueous based formulation of claim 1 wherein said carbon black is present at a level of 1.0×10⁻⁵ wt. % to 1.0×10⁻⁴ wt. %.

12. The aqueous based formulation of claim 1 wherein said carbon black is present at a level of 2.0×10⁻⁵ wt. % to 5.0×10⁻⁵ wt. %.

13. An aqueous based formulation comprising:
0.01 wt. % to 10.0 wt. % humic acid; and
≤5.0 wt. % of activated carbon black.

14. The aqueous based formulation of claim 13 wherein said humic acid is present at a level of 0.01 wt. % to 5.0 wt. %.

15. The aqueous based formulation of claim 13 wherein said humic acid is present at a level of 0.01 wt. % to 3.0 wt. %.

16. The aqueous based formulation of claim 13 wherein said humic acid is present at a level of 0.01 wt. % to 0.10 wt. %.

17. The aqueous based formulation of claim 13 wherein said humic acid is present at a level of 0.03 wt. % to 0.09 wt. %.

18. The aqueous based formulation of claim 13 wherein said carbon black is present at a level of 1.0×10⁻⁵ wt. % to 5.0 wt. %.

19. The aqueous based formulation of claim 13 wherein said carbon black is present at a level of 1.0×10⁻⁵ wt. % to 4.0 wt. %.

20. The aqueous based formulation of claim 13 wherein said carbon black is present at a level of 1.0×10⁻⁵ wt. % to 3.0 wt. %.

21. The aqueous based formulation of claim 13 wherein said carbon black is present at a level of 1.0×10⁻⁵ wt. % to 2.0 wt. %.

22. The aqueous based formulation of claim 13 wherein said carbon black is present at a level of 1.0×10⁻⁵ wt. % to 1.0 wt. %.

23. The aqueous based formulation of claim 13 wherein said carbon black is present at a level of 1.0×10⁻⁵ wt. % to 1.0×10⁻⁴ wt. %.

24. The aqueous based formulation of claim 13 wherein said carbon black is present at a level of 2.0×10⁻⁵ wt. % to 5.0×10⁻⁵ wt. %.
